(19) United States
(12) Patent Application Publication
(10) Pub. No.: US 2007/0224259 A1
(43) Pub. Date: Sep. 27, 2007

(54) ANTI-INFLAMMATORY PHARMACEUTICAL COMPOSITION

(76) Inventors: Anil Kumar Gupta, Kharagpur (IN); Ravinder Kodipyaka, Sirpur-Kaghaznagar (IN); Subhash Pandurang Gore, Solapur (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/533,812

(22) Filed: Sep. 21, 2006

Related U.S. Application Data
(60) Provisional application No. 60/788,820, filed on Apr. 3, 2006.

(30) Foreign Application Priority Data
Sep. 21, 2005 (IN) 1340/CHE/2005

(51) Int. Cl. A61K 9/48; A61K 9/20; A61K 31/557; A61K 31/192

(52) U.S. Cl. 424/451; 424/464; 514/573; 514/570

(57) ABSTRACT

The present invention provides an orally administrable capsule comprising at least two mini-tablets, or at least two mini-capsules, or a combination of at least a mini-tablet and a mini-capsule, wherein one of said mini-tablet or mini-capsule comprises a nonsteroidal anti-inflammatory drug and the other mini-tablet or mini-capsule comprises a prostaglandin.

### INTRODUCTION TO THE INVENTION

The present invention relates to a pharmaceutical dosage form in the form of a capsule comprising at least two smaller tablets or capsules or at least one capsule and at least one tablet, one of which smaller tablets or capsule comprises an NSAID and the other of which smaller tablet or capsule comprises a prostaglandin.

Nonsteroidal anti-inflammatory drugs (“NSAIDs”) comprise a class of drugs having a high therapeutic value especially for the treatment of inflammatory diseases such as osteoarthritis and rheumatoid arthritis.

Diclofenac chemically is 2-[(2,6-dichlorophenyl)amino]benzene acetic acid, with the structural Formula I. It is used in the form of potassium, sodium and diethylamine salts as a cyclooxygenase inhibitor, analgesic and as a non-steroidal anti-inflammatory agent.

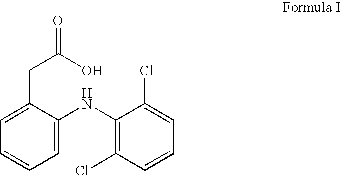

NSAIDs on chronic use have ulcerogenic effects, which can be dangerous. Such ulcers generally exhibit few or no symptoms and may prove to be fatal. Certain prostaglandins such as misoprostol, carboprost, dinoprost, gemeprost, metenoprost, sulprostone, tiaprost and ornoprostil have shown to prevent NSAID induced ulcers. Misoprostol is a prostaglandin, which has been approved for use in the treatment of NSAID induced ulcers.

Misoprostol chemically is (11α,13E)-11,16-dihydroxy-16-methyl-9-oxoprost-13-en-1-oic acid methyl ester (synthetic analog of alprostadil, prostaglandin E₁) and is a cytoprotective prostaglandin PGE₁ analog, with the structural Formula II. It is used in the treatment of benign gastric and duodenal ulceration and in the prevention of NSAID-induced ulcers. It is commercially available as CYTOTEC™, which is an oral tablet containing 100 μg or 200 μg of misoprostol. It is manufactured by G. D. Searle & Co., USA.

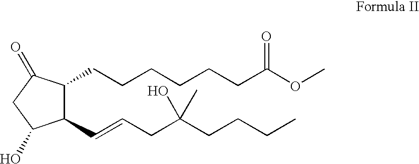

In such cases, wherein an active causes undesirable side effects upon administration exacerbated due to chronic use, the use of another active, which prevents or treats the undesirable side effects, is highly encouraged.

A combination product of diclofenac sodium and misoprostol is commercially available under the brand name ARTHROTEC™, manufactured by G. D. Searle & Co., USA. ARTHROTEC™ is of a core/mantle tablet wherein the outer mantle coating surrounds the inner core. The inner core contains an NSAID, which is further coated with an enteric coating, followed by a mantle coating containing a prostaglandin. Each tablet has an enteric-coated core containing either 50 mg or 75 mg of diclofenac sodium surrounded by an outer mantle containing 200 μg of misoprostol. This product is indicated for the treatment of osteoarthritis and rheumatoid arthritis in patients at high risk of developing NSAID-induced gastric and duodenal ulcers.

U.S. Pat. Nos. 6,787,155, 6,537,582 and 6,387,410 describe an oral pharmaceutical dosage form comprising a hard gelatin capsule filled with a mixture of a delayed release formulation of an NSAID and a mixture containing a prostaglandin.

U.S. Pat. No. 6,287,600 describes a solid composition comprising enterically coated particles of a NSAID, a prostaglandin and a prostaglandin stabilizer.

U.S. Pat. No. 6,740,340 discloses a tablet comprising a shell in which is imbedded two smaller tablets whereby the two smaller tablets are not exposed to the environment.

U.S. Pat. Nos. 5,601,843, 5,698,225 and 5,601,843 describe a tablet comprising a core of an NSAID and a surrounding mantle coat comprising prostaglandin.

U.S. Pat. No. 5,015,481 discloses a stabilized oral and pharmaceutical composition comprising a NSAID, a prostaglandin and hydroxypropyl methylcellulose.

Most of the previous approaches to formulate NSAIDs and prostaglandins together relate to a ‘core and coat’ concept or an admixture of pellets or beads or granules concept. There is a long felt need to develop an economical and industrially viable combination product of a NSAID or pharmaceutically acceptable salts, solvates, enantiomers or mixtures thereof with a prostaglandin, which does not make use of specialized expensive and time-consuming techniques.

### SUMMARY OF THE INVENTION

In an aspect, the invention provides a pharmaceutical dosage form comprising at least two tablets, or at least two capsules, or a combination of at least a tablet and a capsule, wherein one of said tablet or capsule comprises a nonsteroidal anti-inflammatory drug and the other tablet or capsule comprises a prostaglandin. The pharmaceutical dosage form is a capsule that contains a tablet or capsule comprising a nonsteroidal anti-inflammatory drug and a tablet or capsule comprising a prostaglandin.

In one embodiment of present invention, the tablet or capsule comprising an NSAID is enteric coated.

In other embodiment, said capsule of present invention comprises at least two tablets, one comprising diclofenac and the other comprising misoprostol.

An embodiment of the invention provides a pharmaceutical dosage form comprising a nonsteroidal anti-inflammatory drug and a prostaglandin drug, wherein one drug is formulated into a tablet or a capsule, the other drug is separately formulated into a tablet or a capsule, and both formulated drugs are present in an outer capsule.

Another embodiment of the invention provides a pharmaceutical dosage form comprising an outer capsule containing: diclofenac or a salt thereof, formulated into a tablet or a capsule; and misoprostol, separately formulated into a tablet or a capsule.

In a further embodiment, the invention provides a pharmaceutical dosage form comprising an outer capsule containing: diclofenac or a salt thereof, formulated into an enteric coated tablet; and misoprostol, separately formulated into a tablet.

### DETAILED DESCRIPTION OF THE INVENTION

The present invention relates to a pharmaceutical dosage form in the form of a capsule comprising at least two smaller tablets or capsules or at least one capsule and at least one tablet, one of which smaller tablets or capsule comprises an NSAID and the other of which smaller tablet or capsule comprises a prostaglandin.

The NSAIDs that can be used include but are not limited to: propionic acid derivatives like ibuprofen, naproxen, flurbiprofen, fenoprofen, ketoprofen, suprofen, fenbufen and fluprofen; acetic acid derivatives like tolmetin, zomepirac, sulindac and indomethacin; fenamic acid derivatives like mefenamic acid; biphenylcarboxylic acid derivatives like diflunisal and flufenisal; oxicams like piroxicam, sudoxicam and isoxicam; benzeneacetic acid derivatives like diclofenac; COX-2 inhibitors like celecoxib, rofecoxib, meloxicam and nimesulide; and their pharmaceutically acceptable salts, solvates, polymorphs, enantiomers and mixtures.

In one embodiment, diclofenac sodium has been found to be useful in the present invention. This drug will be discussed below in detail to exemplify the invention, but it is to be understood that the invention is not limited to any particular NSAID.

In one embodiment, the present invention provides a unit dose of diclofenac sodium from about 20 to about 100 milligrams or from 40 to about 80 milligrams.

Ulcer protective prostaglandins or their analogues useful in the present invention include but are not limited to misoprostol, carboprost, ornoprostil, dinoprost, gemeprost, metenoprost, sulprostone, tiaprost, and their pharmaceutically acceptable salts or mixtures thereof.

In one embodiment, misoprostol has been found to be useful in the present invention. This drug will be discussed below in detail to exemplify the invention, but it is to be understood that the invention is not limited to any particular prostaglandin.

In an embodiment, the dose of misoprostol per tablet ranges from about 100 to about 300 μg, or from about 150 to about 200 μg.

In one embodiment, the compositions of the present invention can be made as mini-tablets within a capsule or mini-capsules within a capsule.

In an embodiment, the dimensions of a diclofenac mini-tablet and a misoprostol mini-tablet are in the range of about 3 mm to about 7 mm, or about 4 mm to about 6 mm. The two tablets are not necessarily of the same size.

In another embodiment, the pharmaceutical dosage form is in the form of a capsule containing at least two smaller capsules, one of which smaller capsule comprises a mini-tablet, granules, pellets, or a powder comprising an NSAID and the other of which smaller capsule comprises a mini-tablet, granules, pellets, or a powder comprising a prostaglandin.

In yet another embodiment, an enteric coating optionally is used to separate the NSAID from the prostaglandin and to aid in controlling the release of the NSAID. The enteric coating of NSAID tablets, granules, or capsules aids in the prevention of degradation of the prostaglandin caused by contact with the NSAID as well as providing direct delivery of the NSAID in the lower gastrointestinal tract rather than in the stomach.

Further, misoprostol in the present invention can be present in the form of a dispersion in a polymer, such as hydroxypropyl methylcellulose or polyvinylpyrrolidone, which is dried to a powder form. Misoprostol is a viscous liquid and hence is dispersed in solid polymeric carriers, which also act as stabilizers. Such dispersions of misoprostol with hydroxypropyl methylcellulose or povidone are available as powders.

Useful polymers of various grades for the formation of dispersions include, but are not limited to: celluloses such as methylcellulose, carboxymethyl cellulose, hydroxypropyl methylcellulose, cross-linked sodium carboxymethyl cellulose and cross-linked hydroxypropyl cellulose; carboxymethylamide, potassium methacrylate/divinylbenzene copolymer, polymethylmethacrylate, polyhydroxyalkyl methacrylate, polyvinylpyrrolidone, cross-linked polyvinylpyrrolidone, high-molecular weight polyvinylalcohols; gums such as natural gum, agar, agrose, sodium alginate, carrageenan, fucoidan, furcellaran, laminaran, hypnea, eucheums, gum arabic, gum ghatti, gum karaya, gum tragacanth and locust beam gum; hydrophilic colloids such as alginates, carbopol and polyacrylamides; other substances such as arbinoglactan, pectin, amylopectin, gelatin, N-vinyl lactams polysaccharides and the like. Combinations of any two or more of these polymers, and other polymers having the required properties are within the scope of the invention.

A tablet containing a drug will typically also include, along with the drug, tableting excipients.

The pharmaceutical compositions of the present invention in the form of tablets may contain one or more diluents to increase the tablet mass so that it becomes easier for the patient and the caregiver to handle.

Common diluents are microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates, potassium chloride, powdered cellulose, sodium chloride, sorbitol, talc and the like.

The pharmaceutical compositions to be made into tablets may further include a disintegrant to accelerate disintegration of the tablet in the patient's stomach. Disintegrants include but not limited to alginic acid, carboxymethyl cellulose calcium, carboxymethylcellulose sodium (e.g. Ac-Di-Sol®, Primellose®), colloidal silicon dioxide, croscarmellose sodium, crospovidone (e.g. Kollidon® and Polyplasdone®), povidone K-30, guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Suitable enteric-coating polymers include but are not limited to the different grades of anionic polymers of methacrylic acid and methacrylates, such as but not limited to those sold as Eudragit™ L100-55, Eudragit™ L30D-55, Eudragit™ L30D-55, Eudragit™ L100, Eudragit™ S100 and Eudragit™ FS30D. Enteric polymers are resistant to dissolution or decomposition in acidic environments, such as the stomach, but dissolve or decompose in higher pH environments. Various enteric polymers are available for allowing a coated material to pass through the stomach intact, then permitting drug release into a desired pH environment, such as that of the duodenum, jejunum, ileum, or colon.

Any aqueous enteric coating technique such as pan coating, fluid bed coating and the like known to a person skilled in the art falls within the scope of the present invention.

Representative plasticizers for coating are materials such as acetyl alkyl citrates, phosphate esters, phthalate esters, amides, mineral oils, fatty acids and esters thereof with polyethylene glycol, glycerin, triacetin or sugars, fatty alcohols, ethers of polyethylene glycol and vegetable oils. Useful fatty alcohols include cetostearyl alcohol, cetyl alcohol, stearyl alcohol, oleyl alcohol and myristyl alcohol.

The shell of the capsule comprises a suitable physiologically inert material such as hydroxypropyl methylcellulose, gelatin, modified starches and the like.

Pharmaceutical compositions for tabletting and film formation may further include ingredients such as, but not limited to, pharmaceutically acceptable glidants, lubricants, flavoring agents, opacifiers, colorants and other commonly used excipients.

The following examples will further illustrate certain specific aspects and embodiments of the invention in greater detail and are not intended to limit the scope of the invention.

### EXAMPLE 1

### Tablet Composition Containing Diclofenac Sodium

| Ingredients | mg/Tablet |
|---|---|
| Diclofenac sodium | 50 |
| Lactose monohydrate | 13 |
| Microcrystalline cellulose | 12.9 |
| Povidone K-30 | 4.8 |
| Magnesium stearate | 0.9 |
| Water | q.s. |

Manufacturing Process:
1. Diclofenac sodium, lactose monohydrate and microcrystalline cellulose were sifted separately through a 425 μm mesh sieve.
2. Povidone K-30 was dissolved in water to prepare the binder solution.
3. Ingredients of step 1 were wet granulated with the binder solution of step 2.
4. The granules of step 3 were dried at 60° C. for 1 hour in an oven.
5. The oversized granules were milled in a comminuting mill to get granules of a size range about 750-1000 μm.
6. The prepared granules were lubricated by mixing with magnesium stearate.
7. The lubricated granules were compressed in a rotary tablet compression machine using 5.5 mm standard concave punches.

### EXAMPLE 2

### Tablet Composition Containing Misoprostol

| Ingredients | mg/Tablet |
|---|---|
| 1% w/w Misoprostol dispersion in | 20.2 |
| hydroxypropyl methylcellulose | |
| Microcrystalline cellulose | 64.8 |
| Sodium starch glycolate | 4.5 |
| Hydrogenated castor oil | 0.4 |
| Colloidal silicon dioxide | 0.2 |

Manufacturing Process:
1. Misoprostol (1% dispersion in hydroxypropyl methylcellulose), microcrystalline cellulose and sodium starch glycolate were sifted through a 425 μm mesh sieve.
2. The ingredients of step 1 were mixed thoroughly in a blender.
3. The blend of step 2 was lubricated with hydrogenated castor oil and colloidal silicon dioxide.
4. Finally, the lubricated blend of step 3 was compressed in the rotary tablet compression machine using 6 mm standard concave punches.

### EXAMPLE 3

### Pharmaceutical Composition of Diclofenac Sodium and Misoprostol

One tablet of diclofenac sodium (prepared in Example 1) and one tablet of misoprostol (from Example 2) were filled into a size ‘0’ hydroxypropyl methylcellulose capsule.

### EXAMPLE 4

### Composition of Diclofenac Sodium 50 mg Tablet and Misoprostol 200 μg Tablet in a Capsule

| | |
|---|---|
| Ingredients | mg/Tablet |

Diclofenac Core Tablet

| | |
|---|---|
| Diclofenac sodium | 50 |
| Lactose monohydrate | 13 |
| Microcrystalline cellulose | 12.9 |
| Corn starch | 8.4 |
| Povidone K-30 | 5.6 |
| Water | q.s. |
| Magnesium stearate | 0.9 |

Enteric Coating

| | |
|---|---|
| Methacrylic acid copolymer type C | 68.3 |
| Triethyl citrate | 17.1 |
| Glyceryl monostearate | 1.38 |
| Titanium dioxide | 1.38 |
| Isopropyl alcohol | q.s. |

Misoprostol Tablet

| | |
|---|---|
| 1% w/w Misoprostol dispersion in | 20.2 |
| hydroxypropyl methylcellulose | |
| Microcrystalline cellulose | 74.8 |
| Sodium starch glycolate | 5.5 |
| Hydrogenated castor oil | 0.5 |
| Colloidal silicon dioxide | 0.3 |

Manufacturing Process:
1. Diclofenac sodium, lactose monohydrate, microcrystalline cellulose and corn starch were mixed together.
2. Povidone was dissolved in water.
3. Mixture of step 1 was granulated with the binder solution of step 2 by standard wet granulation process.
4. The granules were dried at 60° C. to get loss on drying less than 2%.
5. Dried granules were lubricated and compressed into mini-tablets using 5.5 mm standard concave punches.
6. Core tablets of step 5 were coated with enteric coating dispersion.
7. Misoprostol, microcrystalline cellulose and sodium starch glycolate were mixed by blending.
8. Blend of step 7 was lubricated with hydrogenated castor oil and colloidal silicon dioxide.
9. The lubricated blend of step 8 was compressed in a rotary tablet compression machine using 6 mm standard concave punches.
10. One enteric coated tablet of step 6 and a mini-tablet of step 9 were filled into a size ‘0’ hydroxypropyl methylcellulose capsule.

### EXAMPLE 5

### Composition of Diclofenac Sodium 50 mg Tablet and Misoprostol 200 μg Capsule, in a Capsule

Composition of misoprostol capsule.

| Ingredients | mg/Capsule |
|---|---|
| 1% w/w Misoprostol dispersion in | 20.2 |
| hydroxypropyl methylcellulose | |
| Microcrystalline cellulose | 64.8 |
| Sodium starch glycolate | 4.5 |
| Hydrogenated castor oil | 0.4 |
| Colloidal silicon dioxide | 0.2 |

Manufacturing Process:
1. Misoprostol, microcrystalline cellulose and sodium starch glycolate were sifted through a 425 μm mesh sieve.
2. The ingredients of step 1 were mixed thoroughly in a blender.
3. The blend of step 2 was lubricated with hydrogenated castor oil and colloidal silicon dioxide.
4. Lubricated blend of step 3 was filled into a size ‘2’ hydroxypropyl methylcellulose capsule.
5. One mini-capsule of step 4 and an enteric coated diclofenac tablet of Example 4 (step 6) were filled into a size ‘0’ hydroxypropyl methylcellulose capsule.

### EXAMPLE 6

### Composition of Diclofenac Sodium 75 mg Tablet and Misoprostol 200 μg Tablet, in a Capsule

Composition of diclofenac tablet.

| Ingredients | mg/Tablet |
|---|---|
| +TC,1/ Diclofenac Core Tablet | |
| Diclofenac sodium | 75 |
| Lactose monohydrate | 19.5 |
| Microcrystalline cellulose | 19.5 |
| Corn starch | 12.6 |
| Povidone K-30 | 7.2 |
| Water | q.s. |
| Magnesium stearate | 1.2 |
| +TC,1/ Enteric Coating | |
| ACRYL-EZE ®^{#} | 8.1 |
| Silicone oil | q.s. |
| Water | q.s. |

# ACRYL-EZE is a formulated water-dispersible enteric acrylic coating system, containing the polymer EUDRAGIT® L100-55 and sold by Colorcon, West Point, Pa. U.S.A. The polymer is designed to be removed in the duodenum, about pH 6 and higher.

Manufacturing process was similar to that described in Example 4.

Composition of misoprostol tablet.

| Ingredients | mg/Tablet |
|---|---|
| 1% w/w Misoprostol dispersion in | 20.2 |
| hydroxypropyl methylcellulose | |
| Microcrystalline cellulose | 64.8 |
| Sodium starch glycolate | 4.5 |
| Hydrogenated castor oil | 0.4 |
| Colloidal silicon dioxide | 0.2 |

Manufacturing process was similar to that described in Example 2. A diclofenac tablet and a misoprostol tablet were placed in a size “0” capsule and tested for in vitro drug release.

In vitro Dissolution Data for Diclofenac:

Media: 0.1 N hydrochloric acid (initial 2 hours) and then phosphate buffer pH 6.8

Apparatus: USP type 2 [“Apparatus 2” in Test 711—Dissolution, United States Pharmacopeia 24, United States Pharmacopeial Convention, Inc., Rockville, Md., page 1942 (2000)].

Stirring speed: 50 rpm

Volume: 900 mL phosphate buffer pH 6.8

Temperature: 37.5±0.5° C.

| Time | % Diclofenac Released in |
|---|---|
| (Minutes) | Phosphate Buffer pH 6.8* |
| 0 | 0 |
| 10 | 20 |
| 20 | 47 |
| 30 | 65 |
| 45 | 87 |
| 60 | 93 |

*No diclofenac release was observed in 0.1 N hydrochloric acid, during the initial two hours exposure to the hydrochloric acid solution.

In vitro dissolution data for misoprostol:

Medium: 0.1 N hydrochloric acid

Apparatus: USP type 2.

Stirring speed: 50 rpm

Volume: 500 mL 0.1 N HCl

Temperature: 37.5±0.5° C.

| Time (Minutes) | % Misoprostol Released |
|---|---|
| 0 | 0 |
| 5 | 70 |
| 15 | 84 |
| 30 | 88 |

1. A pharmaceutical dosage form comprising a nonsteroidal anti-inflammatory drug and a prostaglandin drug, wherein one drug is formulated into a tablet or a capsule, the other drug is separately formulated into a tablet or a capsule, and both formulated drugs are present in an outer capsule.

2. The pharmaceutical dosage form of claim 1, wherein a nonsteroidal anti-inflammatory drug comprises diclofenac or a salt thereof.

3. The pharmaceutical dosage form of claim 1, wherein a prostaglandin drug comprises misoprostol.

4. The pharmaceutical dosage form of claim 1, wherein a nonsteroidal antiinflammatory drug is formulated into a tablet that is coated with an enteric polymer.

5. The pharmaceutical dosage form of claim 1, wherein a nonsteroidal antiinflammatory drug is formulated into granules that are coated with an enteric polymer and filled into a capsule.

6. The pharmaceutical dosage form of claim 1, wherein a nonsteroidal anti-inflammatory drug is formulated into a tablet or granules, and filled into an enteric polymer-coated capsule.

7. The pharmaceutical dosage form of claim 1, wherein a nonsteroidal anti-inflammatory drug is formulated into a tablet and a prostaglandin drug is formulated into a tablet.

8. The pharmaceutical dosage form of claim 1, wherein a nonsteroidal anti-inflammatory drug is formulated into a tablet and a prostaglandin drug is formulated into a capsule.

9. The pharmaceutical dosage form of claim 1, wherein a tablet has a diameter about 3 mm to about 7 mm.

10. The pharmaceutical dosage form of claim 1, wherein a tablet has a diameter about 4 mm to about 6 mm.

11. A pharmaceutical dosage form comprising an outer capsule containing:
diclofenac or a salt thereof, formulated into a tablet or a capsule; and
misoprostol, separately formulated into a tablet or a capsule.

12. The pharmaceutical dosage form of claim 11, wherein a tablet or capsule containing diclofenac or a salt thereof is coated with an enteric polymer.

13. The pharmaceutical dosage form of claim 11, wherein diclofenac is formulated as granules that are coated with an enteric polymer and filled into a capsule.

14. The pharmaceutical dosage form of claim 11, wherein a tablet has a diameter about 3 mm to about 7 mm.

15. The pharmaceutical dosage form of claim 11, wherein a tablet has a diameter about 4 mm to about 6 mm.

16. A pharmaceutical dosage form comprising an outer capsule containing:
diclofenac or a salt thereof, formulated into an enteric coated tablet; and
misoprostol, separately formulated into a tablet.

17. The pharmaceutical dosage form of claim 16, wherein diclofenac is present as a salt.

18. The pharmaceutical dosage form of claim 16, wherein a diclofenac tablet is coated to release drug at pH values at least about 6.

19. The pharmaceutical dosage form of claim 16, having a tablet containing about 50 μg of diclofenac and a tablet containing about 200 μg of misoprostol.

20. The pharmaceutical dosage form of claim 16, wherein each tablet has a diameter about 3 mm to about 7 mm.
